(19) United States
(12) Patent Application Publication
(10) Pub. No.: US 2010/0198188 A1
(43) Pub. Date: Aug. 5, 2010

(54) Devices and Methods for Metering Insoluble Active Agent Particles

(75) Inventor: Adam Heller, Austin, TX (US)

Correspondence Address: Abbott Diabetes Care Inc.;Bozicevic, Field & Francis LLP, 1900 University Ave, Suite 200, East Palo Alto, CA, 94303, US

(73) Assignee: Abbott Diabetes Care Inc.

(21) Appl. No.: 12/699,258

(22) Filed: Feb. 3, 2010

Related U.S. Application Data
(60) Provisional application No. 61/150,209, filed on Feb. 5, 2009.
(60) Provisional application No. 61/225,415, filed on Jul. 14, 2009.

(51) Int. Cl. A61M 37/00

(52) U.S. Cl. 604/500; 604/65

(57) ABSTRACT

Embodiments of the present disclosure relate to devices for delivering a predetermined amount of an active agent to a subject by inclusion of a detector configured to detect the number of insoluble active agent particles in a fluid flowing by the detector. A controller is operably connected to the detector and configured to determine the amount of the active agent to be delivered to the subject based on the number of insoluble active agent particles flowing by the detector. Also provided are methods of using the subject devices for delivering a predetermined amount of an active agent to a subject.

### CROSS-REFERENCE TO RELATED APPLICATIONS

Pursuant to 35 U.S.C. §119(e), this application claims priority to the filing date of U.S. Provisional Patent Application Ser. No. 61/150,209, filed Feb. 5, 2009, and U.S. Provisional Patent Application Ser. No. 61/225,415, filed Jul. 14, 2009, both of which are incorporated herein by reference in their entirety.

### INTRODUCTION

The management of diabetes requires the administration of known doses of insulin at defined times, often associated with meals. The dose may vary, for example, with the type of insulin used, with the types and amounts of foods and drinks consumed, and with the time of the day. Insulin may be administered to a subject by injection of a measured volume of an insulin solution, often with an insulin syringe or insulin pen. Insulin may also be administered using an implanted or skin-worn insulin pump. These systems usually administer clear, homogenous insulin solutions.

### SUMMARY

Embodiments of the present disclosure relate to devices for delivering a predetermined amount of an active agent to a subject by inclusion of a detector configured to detect the number of insoluble active agent particles in a fluid flowing by the detector. A controller is operably connected to the detector and configured to determine the amount of the active agent to be delivered to the subject based on the number of insoluble active agent particles flowing by the detector. Also provided are methods of using the subject devices for delivering a predetermined amount of an active agent to a subject.

### BRIEF DESCRIPTION OF THE DRAWINGS

A detailed description of various embodiments of the present disclosure are provided herein with reference to the accompanying drawings, which are briefly described below. The drawings are illustrative and are not necessarily drawn to scale. The drawings illustrate various embodiments of the present disclosure and may illustrate one or more embodiment(s) or example(s) of the present disclosure in whole or in part. A reference numeral, letter, and/or symbol that is used in one drawing to refer to a particular element may be used in another drawing to refer to a like element.

FIG. 1 shows a schematic of a device according to embodiments of the present disclosure.

FIG. 2 shows a schematic of a device configured to detect an electrical characteristic of a fluid flowing by a detector according to embodiments of the present disclosure.

FIG. 3 shows a schematic of a device configured to detect an optical characteristic of a fluid flowing by a detector according to embodiments of the present disclosure.

FIG. 4 shows a block diagram of an embodiment of an analyte monitoring device according to embodiments of the present disclosure.

FIG. 5 shows a block diagram of an embodiment of the primary receiver unit of the analyte monitoring device of FIG. 4.

### DETAILED DESCRIPTION

Before the embodiments of the present disclosure are described, it is to be understood that this invention is not limited to particular embodiments described, as such may, of course, vary. It is also to be understood that the terminology used herein is for the purpose of describing particular embodiments only, and is not intended to be limiting, since the scope of the embodiments of the invention will be limited only by the appended claims.

Where a range of values is provided, it is understood that each intervening value, to the tenth of the unit of the lower limit unless the context clearly dictates otherwise, between the upper and lower limits of that range is also specifically disclosed. Each smaller range between any stated value or intervening value in a stated range and any other stated or intervening value in that stated range is encompassed within the invention. The upper and lower limits of these smaller ranges may independently be included or excluded in the range, and each range where either, neither or both limits are included in the smaller ranges is also encompassed within the invention, subject to any specifically excluded limit in the stated range. Where the stated range includes one or both of the limits, ranges excluding either or both of those included limits are also included in the invention.

In the description of the invention herein, it will be understood that a word appearing in the singular encompasses its plural counterpart, and a word appearing in the plural encompasses its singular counterpart, unless implicitly or explicitly understood or stated otherwise. Merely by way of example, reference to “an” or “the” “analyte” encompasses a single analyte, as well as a combination and/or mixture of two or more different analytes, reference to “a” or “the” “concentration value” encompasses a single concentration value, as well as two or more concentration values, and the like, unless implicitly or explicitly understood or stated otherwise. Further, it will be understood that for any given component described herein, any of the possible candidates or alternatives listed for that component, may generally be used individually or in combination with one another, unless implicitly or explicitly understood or stated otherwise. Additionally, it will be understood that any list of such candidates or alternatives, is merely illustrative, not limiting, unless implicitly or explicitly understood or stated otherwise.

Various terms are described below to facilitate an understanding of the invention. It will be understood that a corresponding description of these various terms applies to corresponding linguistic or grammatical variations or forms of these various terms. It will also be understood that the invention is not limited to the terminology used herein, or the descriptions thereof, for the description of particular embodiments. Merely by way of example, the invention is not limited to particular analytes, bodily or tissue fluids, blood or capillary blood, or sensor constructs or usages, unless implicitly or explicitly understood or stated otherwise, as such may vary.

The publications discussed herein are provided solely for their disclosure prior to the filing date of the application. Nothing herein is to be construed as an admission that the embodiments of the invention are not entitled to antedate such publication by virtue of prior invention. Further, the dates of publication provided may be different from the actual publication dates which may need to be independently confirmed.

### Active Agent Delivery Device

Embodiments of the present disclosure relate to devices for delivering a predetermined amount of an active agent to a subject by inclusion of a detector configured to detect the number of insoluble active agent particles in a fluid flowing by the detector. A controller is operably connected to the detector and configured to determine the amount of the active agent to be delivered to the subject based on the number of insoluble active agent particles flowing by the detector. Also provided are methods of using the subject devices for delivering a predetermined amount of an active agent to a subject.

In certain embodiments, the device is configured to deliver a predetermined amount of an active agent to a subject. By “predetermined” is meant that the amount of active agent to be delivered to the subject is determined before or at substantially the same time as delivery of the active agent to the subject. The amount of active agent to be delivered to the subject may be a specific amount of active agent. For example, the amount of active agent may be a therapeutically effective amount of the active agent. By “effective amount” or “therapeutically effective amount” is meant a dosage sufficient to provide for treatment for the condition or disease state being treated or to otherwise provide the desired effect. The precise dosage will vary according to a variety of factors such as subject-dependent variables (e.g., age, health, etc.), the severity of the condition or disease (e.g., the extent and duration of hyperglycemia), and the treatment being effected. In the case of hyperglycemia, an “effective amount” is that amount necessary to substantially improve the likelihood of treating the hyperglycemia, in particular that amount which improves the likelihood of successfully reducing blood glucose levels to a normal or desired range.

In some instances, the device is configured to meter the amount of active agent to be delivered to the subject. By “meter” is meant to deliver the active agent in a measured amount. For example, if the active agent to be delivered includes insoluble active agent particles suspended in a fluid, then the device may be configured to count the number of insoluble active agent particles to be delivered to the subject.

It will be appreciated that via a fluid, a wide variety of active agents, such as drugs or medicaments, may be delivered to a subject, such as an antibiotic, protein or peptide, a nutritional fluid, a dietary supplement, a health supplement, total parenteral nutrition (TPN), an analgesic, an anesthetic, a pain reliever, such as morphine, for example, a hormone, a hormonal drug, a gene therapy drug, an anticoagulant, a cardiovascular drug, nucleoside analogs, such as azidothymidine (AZT), a chemotherapeutic drug, any source or prodrug thereof, and any combination thereof. In certain embodiments, the active agent is an insoluble active agent. By “insoluble” is meant that the active agent does not substantially dissolve in a liquid solvent, such that the active agent is present in a fluid as a suspension of active agent particles or active agent-containing particles. The insoluble active agent particles may be injected or infused into the subject and dissolve or disintegrate to release the active agent in the subject. In some cases, large peptides and proteins, such as insulin and biological drugs, are more stable when immobilized in particles than when they are dissolved in homogeneous solutions. The greater stability of particles, and lesser likelihood of loss of their activity may be derived, for example, from slower structural changes in and/or slower hydrolysis of the peptide or protein.

In certain embodiments, the insoluble active agent particles are crystalline, such as microcrystalline, nanocrystalline, or the like. In some cases, the insoluble active agent particles are amorphous. The size and shape of the insoluble active agent particles may be substantially uniform or may be non-uniform. In some instances, the insoluble active agent particles have a substantially uniform spherical shape. The average diameter of the insoluble active agent particles may range from 1 μm to 500 μm, such as from 2 μm to 200 μm, including from 5 μm to 50 μm. For example, the insoluble active agent particles may have an average volume from 1 μL to 100 μL, such as from 1 μL to 50 μL, including from 2 μL to 20 μL.

In certain embodiments, the insoluble active agent particles contain the active agent in an amount ranging from 1 wt % to 100 wt % active agent, such as from 10 wt % to 100 wt % active agent, including from 25 wt % to 100 wt % active agent, for example from 50 wt % to 100 wt % active agent. For example, the insoluble active agent particles can include from 1 μg to 200 μg active agent, such as from 1 μg to 100 μg of active agent, including from 3 μg to 20 μg of active agent. The insoluble active agent particles can be suspended or dispersed in a carrier medium, such as a fluid. In some instances, the fluid is a biologically inactive fluid, such as an aqueous poly(ethylene glycol) solution, as described below. The weight fraction of insoluble active agent in the fluid may range from 1 wt % to 50 wt %, such as from 2 wt % to 20 wt %, including from 5 wt % to 15 wt %.

In certain embodiments, the insoluble active agent particles can include fluorescently labeled active agent or a fluorescently labeled carrier associated with the active agent. The fluorescent label can be any type of fluorescent label that is able to be detected by the subject detectors and that does not interfere with the biological activity of the active agent. For example, the fluorescent label can include, for example, the FDA approved fluorescer indocyanine green (ICG) and the like.

In some cases, the active agent is insulin. The term “insulin” is meant to include naturally occurring or recombinant human or animal insulin, including any precursors, analogs, derivatives or prodrugs thereof, as well as any polypeptide that induces the uptake of glucose by cells, and any polypeptide-containing precursor that is converted in the body to an active form of insulin. “Active insulin” is insulin that induces the uptake of glucose by cells. In certain instances, the insulin is insoluble insulin. In certain embodiments, insoluble insulin includes a suspension of insulin-containing particles that may be injected or infused into the subject. The insoluble insulin may dissolve or disintegrate to release active insulin in the subject.

Insoluble insulin includes insoluble particles of insulin and insoluble insulin-containing particles. In some cases, the insoluble particles are composed substantially of insoluble insulin. For example, the insoluble insulin can be acylated insulin, microcrystalline insulin, nanocrystalline insulin, microspherical insulin, nanospherical insulin, and the like. In certain instances, the insoluble insulin includes insoluble insulin-containing particles, which include insoluble particles associated with insulin. For example, the insoluble insulin-containing particles can include biodegradable carriers loaded with insulin. The biodegradable carriers can dissolve, disintegrate or release insulin after being administered to the subject, thus releasing active insulin in the subject.

Examples of insoluble insulin that are useful include, but are not limited to, the following: acylated insulin complexed with zinc, protamine, and a phenolic compound, as described in U.S. Pat. Nos. 6,465,426 and 6,268,335; insoluble insulin particles obtained by milling zinc-insulin in the presence of surfactant F68, sodium deoxycholate, and water at neutral pH, as described by E. Merisko-Liversidge, S. L. McGurk and G. G. Liversidge, “Insulin Nanoparticles: A Novel Formulation Approach for Poorly Water Soluble Zn-Insulin”, Pharmaceutical Research, 21(9), 1545-1553 (2004); insoluble microspherical insulin obtained by cooling preheated insulin-poly(ethylene glycol)-water solutions, as described by L. Bromberg, J. Rashba-Step, and T. Scott, “Insulin Particle Formation in Supersaturated Aqueous Solutions of Poly(Ethylene Glycol)”, Biophysical Journal, 89, 3424-3433 (November 2005); insoluble insulin-containing polyisobutylcyanoacrylate (PIBCA) nanospheres, as described by M. A. Radwan, “Enhancement of Absorption of Insulin-Loaded Polyisobutylcyanoacrylate Nanospheres by Sodium Cholate After Oral and Subcutaneous Administration in Diabetic Rats”, Drug Development and Industrial Pharmacy, 27(9), 981-989 (July 2001); the disclosures of which are incorporated herein by reference in their entirety.

### Detector

In certain embodiments, the device includes a detector. The detector may be configured to detect the number of insoluble active agent particles in a fluid flowing by the detector. The amount of the active agent to be delivered to a subject can be determined based on the number of insoluble active agent particles flowing by the detector. For example, if the amount of active agent contained in an insoluble active agent particle is known, then the amount of the active agent to be delivered to a subject can be determined based on the number of insoluble active agent particles flowing by the detector. In some cases, the detector is configured to detect the number and the size of the insoluble active agent particles in the fluid flowing by the detector. In these cases, the amount of the active agent to be delivered to the subject can be determined based on the number and size of the insoluble active agent particles flowing by the detector. For example, if the amount of active agent contained in an insoluble active agent particle depends on the size of the insoluble active agent particle, then the amount of the active agent to be delivered to a subject can be determined based on the number and size of insoluble active agent particles flowing by the detector.

In certain instances, the detector is configured to detect an electrical characteristic of the fluid flowing by the detector. The detector may be configured to detect an electrical characteristic, such as, but not limited to, the capacitance, the resistance, the conductance, or the impedance of the fluid flowing by the detector. For example, a fluid flowing by the detector may, by itself, have a certain electrical characteristic. If the fluid includes insoluble active agent particles, the electrical characteristic of the fluid may be different. In some cases, the change in the electrical characteristic of the fluid is related to the number of the insoluble active agent particles in the fluid. For instance, the number of insoluble active agent particles in the fluid may be related to the change in capacitance of the fluid as the insoluble active agent particles flow by the detector. In certain instances, the change in the electrical characteristic of the fluid is related to the number and size of the insoluble active agent particles in the fluid. For example, the number and size of the insoluble active agent particles in the fluid may be related to the change in capacitance of the fluid as the insoluble active agent particles flow by the detector.

In embodiments of the detector configured to detect an electrical characteristic of the fluid flowing by the detector, the detector includes one or more electrodes. For example, the detector may include one, two, three, four, five, six, seven, eight, nine, ten, or more electrodes. The electrodes may be configured to detect an electrical characteristic of the fluid, such as, but not limited to, the capacitance, the resistance, the conductance, or the impedance of the fluid as the fluid flows by the detector. Exemplary electrode materials include, but are not limited to, stainless steel, gold, gold-plated substrates, such as gold-plated aluminum, platinum, platinum-plated substrates, such as platinum-plated aluminum, zinc, magnesium, nickel, silicon, carbon fiber, graphite, conductive polymers, combinations thereof, and the like. In some embodiments, the electrodes are not insulated, such that one or more surfaces of the electrode contact the fluid as the fluid flows by the detector. In other embodiments, the electrodes are insulated, such that the surface of the electrode does not come in direct contact with the fluid as the fluid flows by the detector. For example, the electrodes may have an insulation layer disposed over the surface of the electrode. The insulation layer can be any material that provides insulation and/or corrosion resistance to the electrodes and may include a dielectric layer, such as but not limited to, a polymer layer, such as polytetrafluoroethylene (PTFE), polyethylene, polyetheretherketone (PEEK), polyether block amide (PEBA), polyamide, and the like.

In other embodiments, the detector is configured to detect an optical characteristic of the fluid flowing by the detector. For example, the detector may be configured to detect the fluorescence of the insoluble active agent particles in the fluid flowing by the detector. In certain cases, the detector is configured to detect the effect the insoluble active agent particles have on electromagnetic radiation directed at the fluid flowing by the detector. For instance, the detector may be configured to detect an optical characteristic, such as, but not limited to, the scattering or the blocking of electromagnetic radiation by the insoluble active agent particles in the fluid flowing by the detector. The electromagnetic radiation may include visible light, such as a laser. In some instances, the detector includes a photodetector, such as, but not limited to, a photodiode, a charge-coupled device (CCD), an intensified charge-coupled device (ICCD), a complementary metal-oxide-semiconductor (CMOS) sensor, and the like.

Embodiments of the detector may also be operably connected to a controller, as described in more detail below. In some instances, the detector includes a transmitter. In certain embodiments, the detector is configured to automatically transmit, e.g., wirelessly or using a physical wired connection, information to the controller, such as signals representing the number and/or size of the insoluble active agent particles flowing by the detector.

### Controller

As described above, in certain embodiments, the device is configured to deliver a predetermined amount of an active agent to a subject. The device may include a controller operably connected to the detector and configured to determine the amount of active agent to be delivered to the subject. The controller may be configured to receive signals from the detector. In some cases, the signals from the detector represent the number of insoluble active agent particles that flow by the detector, as described above. In these cases, the controller may determine the amount of active agent to be delivered to the subject based on the number of insoluble active agent particles that flow by the detector. In certain instances, the signals from the detector represent the number and size of insoluble active agent particles that flow by the detector, as described above. In these instances, the controller may determine the amount of active agent to be delivered to the subject based on the number and size of insoluble active agent particles that flow by the detector.

If the amount of active agent to be delivered to the subject is successfully determined, it may be displayed, stored, and/or otherwise processed to provide useful information. By way of example, a raw signal representing the number and/or number and size of insoluble active agent particles may be used as a basis for determining the amount of active agent. The controller may be configured to monitor the number and/or number and size of the insoluble active agent particles over a period of time. In some instances, the period of time corresponds to the amount of time it takes to deliver the desired dose of active agent to the subject. The period of time may correspond to the delivery of a bolus of active agent to the subject. For example, the period of time can range from 0.1 sec to 60 min, such as from 0.5 sec to 30 min, including from 1 sec to 10 min, or from 1 sec to 1 min. In some cases, the period of time corresponds to the delivery of a basal rate of active agent to the subject over a longer period of time. For example, the period of time may be 1 hour or greater, such as 2 hours or greater, 4 hours or greater, 8 hours or greater, 12 hours or greater, 24 hours or greater, including 2 days or greater, 4 days or greater, 7 days or greater, for instance 2 weeks or greater, 3 weeks or greater, 4 weeks or greater, etc.

The controller may be configured to store the cumulative number of insoluble active agent particles that flow by the detector over a period of time. In some cases, the controller is configured to store information related to the size of the insoluble active agent particles that flow by the detector over a period of time. The information related to the size of the insoluble active agent particles can include, but is not limited to, the size of each insoluble active agent particle that flows by the detector, an average of the sizes of the insoluble active agent particles that flow by the detector over a period of time, and the like.

The controller may be operably associated with a display device, a recording device, a printing device, and/or any device sufficient for communicating information concerning the number of insoluble active agent particles, the size of insoluble active agent particles, the amount of active agent, and the like. The controller may be operably connected to a pump for feedback control, such as to adjust a pumping parameter such as flow volume, flow rate, pulse frequency, or pulse duration, for example, based on the number of insoluble active agent particles, the size of insoluble active agent particles and/or the amount of active agent flowing by the detector.

The controller may further include any appropriate components configured to achieve any of its intended purposes. Examples of the components include, but are not limited to, any one or more of the following: electronic circuitry; componentry; storage media; signal- or data-processing elements; algorithmic elements; software elements; logic devices; wired or wireless communication elements; devices for operable communication between elements or components; and the like. Examples of the intended purposes the components may be configured to achieve include, but are not limited to, any one or more of the following: providing a potential or a current to an electrode; obtaining information from the detector; obtaining information from a fluid delivery device; obtaining information from a temperature probe; obtaining information pertinent to calibration and/or control of a fluid delivery device; assessing or processing any obtained or internal information; communicating with the detector or another device, such as a fluid delivery device, a display device, an alarm or notification device, and/or a calibration device, such as an autocalibration device; communicating via a feedback loop, such as an automated feedback loop; and calibrating and/or controlling another device, such as a fluid delivery device. The controller may be configured to include any suitable elements described herein, or any suitable elements for achieving any of the purposes described herein. Any device with which the controller may communicate may be equipped with complementary elements, such as any suitable communication elements, components, or devices, such as, but not limited to, wired or wireless communication elements, such as a transmitter and/or a receiver. In certain embodiments, the communication elements are configured to use one or more of the following: an RF communication protocol, an infrared communication protocol, a Bluetooth enabled communication protocol, an 802.11x wireless communication protocol, or an equivalent wireless communication protocol which would allow secure, wireless communication of several units (for example, per Health Insurance Portability and Accountability Act (HIPPA) requirements), while avoiding potential data collision and interference.

In some instances, the controller includes or is operably connected to a user interface. The user interface may be configured to receive inputs from a user, such as setting one or more operating parameter(s), such as a desired dose of active agent, an activation or deactivation of the fluid delivery device, and the like, and any combination thereof.

### Reservoir

In certain embodiments, the device includes a source of a fluid, such as a fluid reservoir.

The reservoir may be configured to contain a fluid that includes the insoluble active agent particles, as described above. The insoluble active agent particles contained in the reservoir may be suspended or dispersed in the fluid, as described above. In some cases, the reservoir is dimensioned to contain a volume of the fluid that corresponds to one or more doses of active agent. In certain embodiments, the volume of the reservoir ranges from 0.1 mL to 100 mL, such as from 0.1 mL to 50 mL, including from 0.1 mL to 25 mL, or from 0.1 mL to 10 mL, or from 0.1 mL to 5 mL, including from 0.1 mL to 1 mL. For example, the reservoir may be configured to include from 1 mg to 500 mg of insoluble active agent particles, such as from 2 mg to 250 mg of insoluble active agent particles, including from 5 mg to 100 mg of insoluble active agent particles, for instance from 10 mg to 50 mg of insoluble active agent particles.

In certain embodiments, the reservoir is in fluid communication with an infusion set and a fluid delivery device, as described in more detail below. The reservoir may be in fluid communication with one or more conduits configured to carry the fluid and deliver the fluid to a subject. For example, the conduit may include tubing in fluid communication with the reservoir and one or more components of the device, such as, but not limited to, a fluid delivery device, an infusion set, and the like.

### Pump

Embodiments of the disclosed devices may also include a fluid delivery device, such as a fluid pump. The fluid delivery device may be in fluid communication with one or more conduits configured to deliver the fluid to a subject. The pump may be configured to deliver the fluid in a continuous manner, or in a non-continuous manner, such as in one or more periodic discrete pulses, two or more periodic discrete pulses, three or more periodic discrete pulses, etc.

Appropriate pump parameters may vary according to the fluid to be pumped and the application of the fluid. For example, in the case of the delivery of a fluid that includes an active agent, a nutrient, a dietary or health supplement, any source thereof, any combination thereof, and the like, to a subject, such parameters may vary according to the active agent, nutrient, or supplement, etc., the characteristics of the subject, the characteristics of the condition the active agent, nutrient, or supplement, etc., is intended to address, and the like.

In certain embodiments, the pump is a micro-pump configured to deliver a fluid in small quantities. In some cases, the pump is configured to deliver fluid in quantities, such as from 1 μL to 10,0000 μL per day, including from 10 μL to 5,0000 μL per day, or from 100 μL to 1,0000 μL per day. For example, the pump may be configured to deliver a fluid in a quantity of 400 μL per day on average, as may be suitable for the delivery of insulin to a diabetic person. The pump may include a micro-pump configured to deliver a fluid in periodic pulses. In some cases, the pump is configured to deliver the fluid in periodic pulses, as may be suitable for the delivery of insulin to a diabetic person. For instance, the pump may be configured to deliver the fluid in periodic pulses, such as from 0.1 μL to 10 μL per pulse, including from 0.1 μL to 5 μL per pulse, or from 0.5 μL to 1 μL per pulse. Suitable pulse periods and repetition rates may vary depending on the application. In certain embodiments, the pump is configured to deliver the fluid at a repetition rate, as may be suitable for the delivery of insulin to a diabetic person. For example, the pump may be configured to deliver the fluid at a repetition rate of from every second for a fast multi-dose bolus to a rate of every 10 minutes for a slow basal rate delivery of active agent. In certain instances, the number of insoluble active agent particles pumped per second ranges from 0 particles/sec to 50,000 particles/sec, such as from 100 particles/sec to 40,000 particles/sec, including from 500 particles/sec to 30,000 particles/sec.

The pump may be operably connected to a controller. In certain instances, the controller is configured for feedback control, such as, but not limited to, adjusting a pumping parameter such as flow volume, flow rate, pulse frequency, and pulse duration. For example, the controller may be configured to adjust the pumping parameter based on signals from the detector concerning the number and/or number and size of insoluble active agent particles flowing by the detector.

The pump may be an implantable pump or external pump. Where the pump is an external pump, the pump may be configured to be attachable to the user or article of clothing of the user, e.g., wearable on a belt (e.g., in the manner of a pager, cellular telephone, or the like) or other article of clothing. The pump may include a processor operably connected to the pump and configured to control the pump. For example, the pump may be configured to pump insulin from the reservoir that contains the insulin to the subject, and may include a processor for determining an appropriate dose of insulin based on certain inputs such as glucose levels, food intake, insulin levels, etc. Accordingly, the pump may be configured to controllably deliver insulin to the subject under the direction of the processor and/or the patient or healthcare provider. The pump may be operably connected to a glucose meter for determining the level of glucose from a test strip or continuous glucose sensor. For example, a test strip port may be included for receiving a test strip. A test strip may be used to determine the subject's blood glucose level prior to insulin delivery, e.g., to confirm a previously obtained glucose level as a safeguard measure.

The fluid delivery device may be of any suitable type, such as a fluid pump, configured to deliver a fluid from an outlet from the fluid delivery device to a conduit configured to carry the fluid. For example, the fluid delivery device may be a fluid pump that includes a shape memory actuator, a piezoelectric pump, and the like. Information concerning the construction and the operation of such a fluid delivery device may be found in U.S. Pat. No. 6,916,159 and U.S. Application Publication No. 2005/0238503, the disclosures of which are incorporated herein by reference in their entirety. In certain embodiments, the fluid delivery device has automatic calibration and/or control capability. Information concerning the construction and the operation of such a fluid delivery device may be found in U.S. Application Publication No. 2005/0235732, the disclosure of which is incorporated herein by reference in its entirety.

### Infusion Set

Embodiments of the subject device may also include an infusion set. “Infusion set” is meant to include any structure or combination of structures that is used to deliver an active agent from a source to a subject. The infusion set may be configured to infuse any fluid that includes an active agent. In some embodiments, the infusion set is in fluid communication with the reservoir and the pump, as described in detail above. For example, the infusion set may be configured to infuse insulin and is in fluid communication with an insulin pump and an insulin reservoir. In some cases, the infusion set includes a securement element configured to attach the infusion set to a surface of the skin of the user.

The infusion set may include an insertion element configured such that, during use, at least a portion of the insertion element is inserted subcutaneously into a subject. For example, the insertion element can include a cannula, needle, and the like. The insertion element can be in fluid communication with a conduit configured to carrying a fluid, such as, but not limited to, a tubing. One end of the tubing may be in fluid communication with the insertion element and the other end of the tubing may be in fluid communication with the pump and/or the reservoir, as described above.

In certain embodiments, the infusion set is included in a separate housing from the pump and reservoir. For example, the infusion set may be included in a first housing and the pump and reservoir may be included in a second housing. The infusion set included in the first housing and the pump and reservoir included in the second housing may be in fluid communication via the tubing. The pump and reservoir may be carried or worn by the user at a separate location from the infusion set. In other embodiments, the infusion set is included in the same housing as the pump and reservoir, such that the infusion set, pump and reservoir are included in a single unit that may be worn by the user or implanted in the user. Accordingly, in such embodiments, a user need only attach a single unit on the user's body, rather than wearing a separate unit for the infusion set and a separate unit for the pump and reservoir.

Aspects of the subject infusion set include embodiments where the detector is included in the infusion set. The detector may be positioned at any desirable position in the fluid path of the fluid as the fluid flows through the device. For example, the detector may be positioned at one or more of the following positions: proximal, such as downstream, from an outlet on the reservoir; proximal to an inlet or an outlet on the pump; at a position along tubing that is in fluid communication with the insertion element and/or the pump and reservoir; proximal to an inlet on the infusion set; and proximal to an outlet on the infusion set, such as included in an insertion element of the infusion set. The infusion set may include an upstream end and a downstream end. By “upstream” is meant at a position along the fluid path that is closer to the source of the fluid. By “downstream” is meant at a position along the fluid path that is further away from the source of the fluid. In certain embodiments, the detector is positioned at a position in the fluid path that facilitates detecting the number of insoluble active agent particles that are delivered to the subject and minimizes detecting insoluble active agent particles that are not delivered to the subject. For example, in some cases, the detector may be positioned proximal to the upstream end of the infusion set, such as proximal to the upstream end of the insertion element (e.g., cannula). In some instances, the detector is positioned proximal to the downstream end of the infusion set, such as proximal to the downstream end of the insertion element (e.g., cannula).

The subject infusion set may be used with an analyte monitoring device using a sensor at least a portion of which is positioned beneath the skin of the user for the in vivo determination of a concentration of an analyte. The sensor may be, for example, subcutaneously positioned in a subject for the continuous or periodic monitoring an analyte in a subject's interstitial fluid. This may be used to infer the glucose level in the subject's bloodstream. Sensors for insertion into a vein, artery, or other portion of the body containing fluid, may also be used. A sensor may be configured for monitoring the level of the analyte over a time period which may range from seconds, minutes, hours, days, weeks, or longer. For example, a continuous glucose monitoring device may be used with an insulin delivery pump and infusion set.

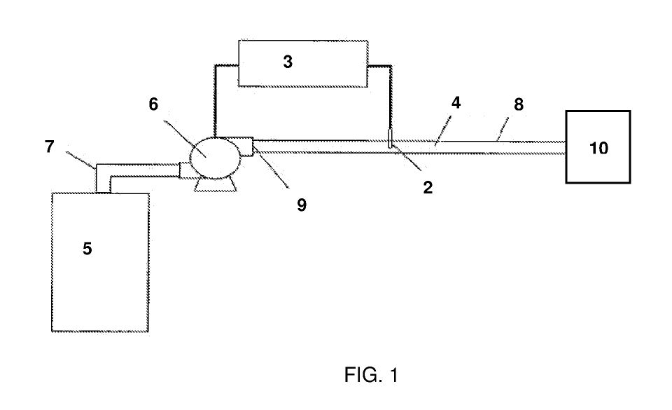

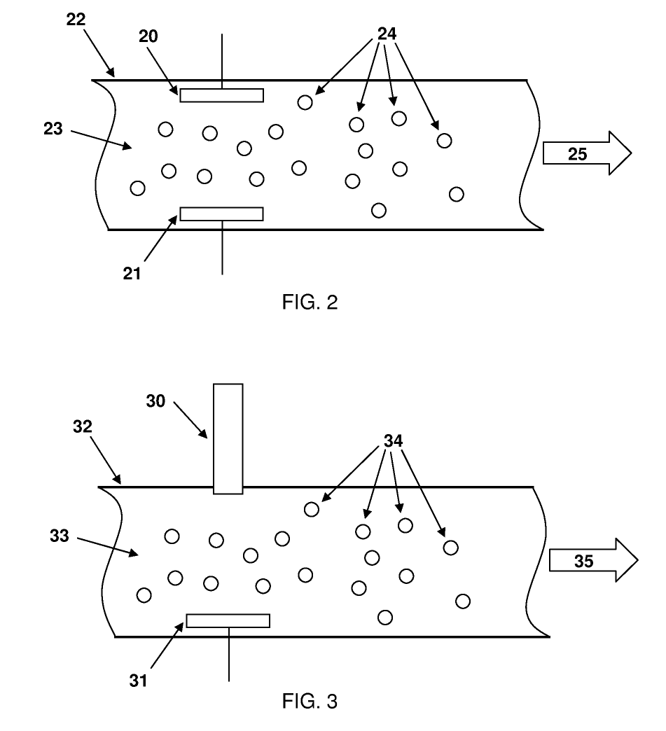

FIG. 1 shows a schematic of a device according to embodiments of the present disclosure. The device includes a detector 2 configured to detect the number of insoluble active agent particles in a fluid 4 flowing by the detector 2. The device also includes a controller 3 operably connected to the detector 2 and configured to determine the amount of the active agent to be delivered to the subject based on the number and/or size of insoluble active agent particles flowing by the detector 2. Also shown in FIG. 2 is a source of fluid, such as a fluid reservoir 5, and a fluid delivery device, such as a pump 6. The fluid reservoir 5 and the pump 6 are in fluid communication, such as via a conduit 7, as shown in FIG. 1. The pump 6 is also in fluid communication with a flow path, such as tubing 8, for the delivery of fluid 4. The flow path 8 is in fluid communication with an outlet 9 of the pump 6. The pump 6 is also operably connected to the controller 3. The tubing 8 is in fluid communication with infusion set 10, which may be attached to a surface of the skin of a user.

FIG. 2 shows a schematic of a device configured to detect an electrical characteristic of a fluid flowing by a detector according to embodiments of the present disclosure. The device includes a detector that includes a first electrode 20 and a second electrode 21. The first electrode 20 and second electrode 21 are positioned in a fluid path 22. Fluid path 22 may be a tubing in fluid communication with a pump and an infusion set (see FIG. 1), or fluid path 22 may be part of an infusion set, such as a cannula. Fluid path 22 contains a fluid 23 as the fluid 23 flows through the fluid path 22 in a downstream direction (indicated by arrow 25). Fluid 23 includes a suspension of insoluble active agent particles 24, such as insoluble insulin particles. The detector is configured to detect transient changes in an electrical characteristic of the fluid 23 due to the insoluble active agent particles 24 as they flow by the first electrode 20 and the second electrode 21 of the detector.

FIG. 3 shows a schematic of a device configured to detect an optical characteristic of a fluid flowing by a detector according to embodiments of the present disclosure. The device includes an electromagnetic radiation source, such as a laser 30. The device also includes a detector 31. The detector 31 is positioned in a fluid path 32. Fluid path 32 may be a tubing in fluid communication with a pump and an infusion set (see FIG. 1), or fluid path 32 may be part of an infusion set, such as a cannula. Fluid path 32 contains a fluid 33 as the fluid 33 flows through the fluid path 32 in a downstream direction (indicated by arrow 35). Fluid 33 includes a suspension of insoluble active agent particles 34, such as insoluble insulin particles. The detector is configured to detect transient changes in an optical characteristic of the fluid 33 due to the insoluble active agent particles 34 as they flow by the detector 31.

### Active Agent Delivery System

The subject active agent delivery device may be included in an active agent delivery system. The active agent delivery system may also include one or more sensors used in sensor-based active agent delivery systems. The active agent delivery system may provide an active agent to counteract a high or low level of an analyte in response to signals from the one or more sensors. In some cases, the active agent delivery system monitors the active agent concentration to ensure that the active agent remains within a desired therapeutic range. The active agent delivery system may include one or more (e.g., two or more) sensors, a processing unit, a transmitter, a receiver/display unit, and an active agent delivery system. In some cases, some or all components may be integrated in a single unit. A sensor-based active agent delivery system may use data from the one or more sensors to provide necessary input for a control algorithm/mechanism to adjust the delivery of the active agent, e.g., automatically or semi-automatically. As an example, a glucose sensor may be used to control and adjust the delivery of insulin from an external or implanted insulin pump.

### Analyte Monitoring Device

In some embodiments, the active agent delivery system includes an analyte monitoring device. The analyte monitoring device may include a sensor control unit and a sensor. In these embodiments, the processing circuit of the sensor control unit is configured to determine a level of an analyte and activate an alarm system if the analyte level exceeds a threshold or is outside a desired range. The sensor control unit may include an alarm system and may also include a display, such as an LCD or LED display.

Representative embodiments of the present disclosure relate to methods and devices for detecting at least one analyte, including glucose, in body fluid. Embodiments relate to the continuous and/or automatic in vivo monitoring of the level of one or more analytes using a continuous analyte monitoring device that includes an analyte sensor at least a portion of which is to be positioned beneath a skin surface of a user for a period of time and/or the discrete monitoring of one or more analytes using an in vitro blood glucose (“BG”) meter and an analyte test strip. Embodiments include combined or combinable devices, systems and methods and/or transferring data between an in vivo continuous system and an in vivo system. In some embodiments, the systems, or at least a portion of the systems, are integrated into a single unit.

A sensor may be an in vivo sensor or an in vitro sensor (i.e., a discrete monitoring test strip), such as an electrochemical sensor that includes one or more of a working electrode, a counter electrode and a reference electrode. The sensor can be formed on a substrate, e.g., a substantially planar substrate.

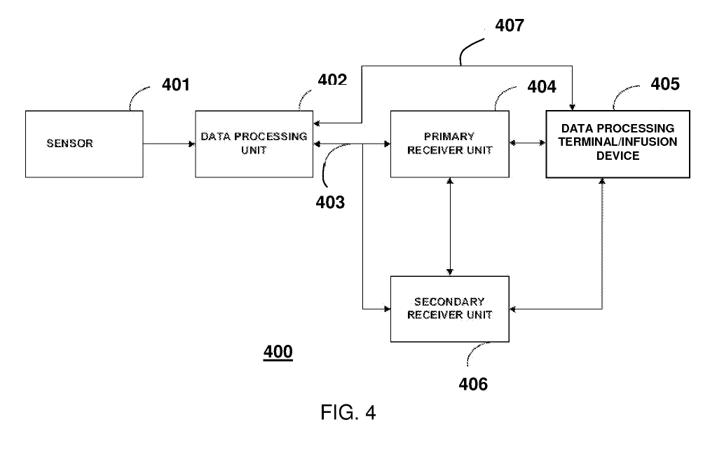

Representative embodiments of test strips include, e.g., FreeStyle® and Precision® blood glucose test strips from Abbott Diabetes Care, Inc. (Alameda, Calif.). Glucose information obtained by the in vitro glucose testing device may be used for a variety of purposes, computations, etc. For example, the information may be used to calibrate the sensor 401 (see FIG. 4), confirm results from the sensor 401 to increase the confidence thereof (e.g., in instances in which information obtained by the sensor is employed in therapy related decisions), etc. In addition to the embodiments specifically disclosed herein, the devices and methods of the present disclosure can be configured to work with a wide variety of analyte sensors, e.g., those disclosed in U.S. patent application Ser. No. 11/461,725, filed Aug. 1, 2006; U.S. Patent Application Publication No. 2007/0095661; U.S. Patent Application Publication No. 2006/0091006; U.S. Patent Application Publication No. 2006/0025662; U.S. Patent Application Publication No. 2008/0267823; U.S. Patent Application Publication No. 2007/0108048; U.S. Patent Application Publication No. 2008/0102441; U.S. Patent Application Publication No. 2008/0066305; U.S. Patent Application Publication No. 2007/0199818; U.S. Patent Application Publication No. 2008/0148873; U.S. Patent Application Publication No. 2007/0068807; U.S. Pat. No. 6,616,819; U.S. Pat. No. 6,143,164; and U.S. Pat. No. 6,592,745; the disclosures of each of which are incorporated herein by reference in their entirety. Additional analyte sensors are described in U.S. patent application Ser. No. 12/102,374, filed Apr. 14, 2008, and U.S. Patent Application Publication No. 2009/0095625, the disclosures of each of which are incorporated herein by reference in their entirety.

Accordingly, embodiments include analyte monitoring devices that include an analyte sensor at least a portion of which is positionable beneath the skin surface of the user for the in vivo detection of an analyte, including glucose, lactate, and the like, in a body fluid. The analyte monitoring system may be configured to monitor a variety of analytes at the same time or at different times. Embodiments include wholly implantable analyte sensors and analyte sensors in which only a portion of the sensor is positioned under the skin and a portion of the sensor resides above the skin, e.g., for contact to a sensor control unit (which may include a transmitter), a receiver/display unit, transceiver, processor, etc. The sensor may be, for example, subcutaneously positionable in a user for the continuous or periodic monitoring of a level of an analyte in the user's interstitial fluid. For the purposes of this description, continuous monitoring and periodic monitoring will be used interchangeably, unless noted otherwise. The sensor response may be correlated and/or converted to analyte levels in blood or other fluids. In certain embodiments, an analyte sensor is positioned in contact with interstitial fluid to detect the level of glucose, which detected glucose may be used to infer the glucose level in the user's bloodstream. Analyte sensors may be insertable into a vein, artery, or other portion of the body containing fluid. Representative embodiments of the analyte sensors having a self-polymerizing hydrogel may be configured for monitoring the level of the analyte over a time period which may range from seconds, minutes, hours, days, weeks, to months, or longer.

In certain embodiments, the analyte sensors, such as glucose sensors, are capable of in vivo detection of an analyte for one hour or more, e.g., a few hours or more, e.g., a few days or more, e.g., three or more days, e.g., five days or more, e.g., seven days or more, e.g., several weeks or at least one month or more. Future analyte levels may be predicted based on information obtained, e.g., the current analyte level at time t₀, the rate of change of the analyte, etc. Predictive alarms may notify the user of a predicted analyte level that may be of concern in advance of the user's analyte level reaching the future level. This provides the user an opportunity to take corrective action.

FIG. 4 shows a data monitoring and management system such as, for example, an analyte (e.g., glucose) monitoring system 400 in accordance with certain embodiments. Embodiments of the subject disclosure are further described primarily with respect to glucose monitoring devices and systems, and methods of glucose detection, for convenience only and such description is in no way intended to limit the scope of the representative embodiments. It is to be understood that the analyte monitoring system may be configured to monitor a variety of analytes at the same time or at different times.

Analytes that may be monitored include, but are not limited to, acetyl choline, amylase, bilirubin, cholesterol, chorionic gonadotropin, creatine kinase (e.g., CK-MB), creatine, creatinine, DNA, fructosamine, glucose, glutamine, growth hormones, hormones, ketone bodies, lactate, peroxide, prostate-specific antigen, prothrombin, RNA, thyroid stimulating hormone, and troponin, or the concentration of active agents, such as, for example, antibiotics (e.g., gentamicin, vancomycin, and the like), digitoxin, digoxin, drugs of abuse, theophylline, warfarin, and the like. In embodiments that monitor more than one analyte, the analytes may be monitored at the same or different times.

As shown in FIG. 4, the analyte monitoring device 400 may include an analyte sensor 401, a data processing unit 402 operably connected to the sensor 401, and a primary receiver unit 404. In some instances, the primary receiver unit 404 is configured to communicate with the data processing unit 402 via a communication link 403. In certain embodiments, the primary receiver unit 404 may be further configured to transmit data to a data processing terminal 405 to evaluate or otherwise process or format data received by the primary receiver unit 404. The data processing terminal 405 may be configured to receive data directly from the data processing unit 404 via a communication link 407, which may optionally be configured for bi-directional communication. Further, the data processing unit 402 may include a transmitter or a transceiver to transmit and/or receive data to and/or from the primary receiver unit 404 and/or the data processing terminal 405 and/or optionally a secondary receiver unit 406.

Also shown in FIG. 4 is an optional secondary receiver unit 406 which is operably coupled to the communication link 403 and configured to receive data transmitted from the data processing unit 402. The secondary receiver unit 406 may be configured to communicate with the primary receiver unit 404, as well as the data processing terminal 405. In certain embodiments, the secondary receiver unit 406 may be configured for bi-directional wireless communication with each of the primary receiver unit 404 and the data processing terminal 405. In some instances, the secondary receiver unit 406 may be a de-featured receiver as compared to the primary receiver unit 404, e.g., the secondary receiver unit 406 may include a limited or minimal number of functions and features as compared with the primary receiver unit 404. As such, the secondary receiver unit 406 may include a smaller (in one or more, including all, dimensions), compact housing or embodied in a device including, for example, a wrist watch, arm band, PDA, mp3 player, cell phone, etc. Alternatively, the secondary receiver unit 406 may be configured with the same or substantially similar functions and features as the primary receiver unit 404. The secondary receiver unit 406 may include a docking portion configured to mate with a docking cradle unit for placement by, e.g., the bedside for night time monitoring, and/or a bi-directional communication device. A docking cradle may recharge a power supply.

Only one analyte sensor 401, data processing unit 402 and data processing terminal 405 are shown in the embodiment of the analyte monitoring device 400 shown in FIG. 4. In certain embodiments, the analyte monitoring device 400 includes one or more sensors 401, and/or one or more data processing units 402, and/or one or more data processing terminals 405. Multiple sensors may be positioned in a user for analyte monitoring at the same or different times, or at different locations in the user. In certain embodiments, analyte information obtained by a first sensor positioned in a user may be employed as a comparison to analyte information obtained by a second sensor. This may be useful to confirm or validate analyte information obtained from one or both of the sensors. Such redundancy may be useful if analyte information is contemplated in critical therapy-related decisions. In certain embodiments, a first sensor may be used to calibrate a second sensor.

The analyte monitoring device 400 may be a continuous monitoring system, or semi-continuous, or a discrete monitoring system. In a multi-component system, each component may be configured to be uniquely identified by one or more of the other components in the system so that communication conflict may be readily resolved between the various components within the analyte monitoring device 400. For example, unique IDs, communication channels, and the like, may be used.

It should be understood that the subject devices and methods may be used in conjunction with stand-alone meters, as well those operably connected to, e.g., integrated with, analyte monitoring systems including implanted or partially implanted analyte sensors (e.g., continuous analyte monitoring systems). Exemplary sensors and meters and continuous analyte monitoring systems (sometimes referred to as in vivo systems) that may be utilized in connection with the disclosed devices and methods include sensors and meters such as those described in U.S. Pat. No. 7,041,468; U.S. Pat. No. 5,356,786; U.S. Pat. No. 6,175,752; U.S. Pat. No. 6,560,471; U.S. Pat. No. 5,262,035; U.S. Pat. No. 6,881,551; U.S. Pat. No. 6,121,009; U.S. Pat. No. 7,167,818; U.S. Pat. No. 6,270,455; U.S. Pat. No. 6,161,095; U.S. Pat. No. 5,918,603; U.S. Pat. No. 6,144,837; U.S. Pat. No. 5,601,435; U.S. Pat. No. 5,822,715; U.S. Pat. No. 5,899,855; U.S. Pat. No. 6,071,391; U.S. Pat. No. 6,120,676; U.S. Pat. No. 6,143,164; U.S. Pat. No. 6,299,757; U.S. Pat. No. 6,338,790; U.S. Pat. No. 6,377,894; U.S. Pat. No. 6,600,997; U.S. Pat. No. 6,773,671; U.S. Pat. No. 6,514,460; U.S. Pat. No. 6,592,745; U.S. Pat. No. 5,628,890; U.S. Pat. No. 5,820,551; U.S. Pat. No. 6,736,957; U.S. Pat. No. 4,545,382; U.S. Pat. No. 4,711,245; U.S. Pat. No. 5,509,410; U.S. Pat. No. 6,540,891; U.S. Pat. No. 6,730,200; U.S. Pat. No. 6,764,581; U.S. Pat. No. 6,299,757; U.S. Pat. No. 6,461,496; U.S. Pat. No. 6,503,381; U.S. Pat. No. 6,591,125; U.S. Pat. No. 6,616,819; U.S. Pat. No. 6,618,934; U.S. Pat. No. 6,676,816; U.S. Pat. No. 6,749,740; U.S. Pat. No. 6,893,545; U.S. Pat. No. 6,942,518; U.S. Pat. No. 6,514,718; U.S. Pat. No. 5,264,014; U.S. Pat. No. 5,262,305; U.S. Pat. No. 5,320,715; U.S. Pat. No. 5,593,852; U.S. Pat. No. 6,746,582; U.S. Pat. No. 6,284,478; U.S. Pat. No. 7,299,082; U.S. Patent Application No. 61/149,639, entitled “Compact On-Body Physiological Monitoring Device and Methods Thereof”, U.S. patent application Ser. No. 11/461,725, filed Aug. 1, 2006, entitled “Analyte Sensors and Methods”; U.S. patent application Ser. No. 12/495,709, filed Jun. 30, 2009, entitled “Extruded Electrode Structures and Methods of Using Same”; U.S. Patent Application Publication No. 2004/0186365; U.S. Patent Application Publication No. 2007/0095661; U.S. Patent Application Publication No. 2006/0091006; U.S. Patent Application Publication No. 2006/0025662; U.S. Patent Application Publication No. 2008/0267823; U.S. Patent Application Publication No. 2007/0108048; U.S. Patent Application Publication No. 2008/0102441; U.S. Patent Application Publication No. 2008/0066305; U.S. Patent Application Publication No. 2007/0199818; U.S. Patent Application Publication No. 2008/0148873; and U.S. Patent Application Publication No. 2007/0068807; the disclosures of each which are incorporated herein by reference in their entirety.

In certain embodiments, the sensor 401 is physically positioned in or on the body of a user whose analyte level is being monitored. The sensor 401 may be configured to at least periodically sample the analyte level of the user and convert the sampled analyte level into a corresponding signal for transmission by the data processing unit 402. The data processing unit 402 may be operably coupled to the sensor 401 so that both devices are positioned in or on the user's body, with at least a portion of the analyte sensor 401 positioned transcutaneously. The data processing unit 402 may include a fixation element, such as an adhesive or the like, to secure the data processing unit 402 to the user's body. A mount (not shown) attachable to the user and mateable with the data processing unit 402 may be used. For example, the mount may include an adhesive surface. The data processing unit 402 may be configured to perform data processing functions, where such functions may include, but are not limited to, filtering and encoding of data signals, each of which corresponds to a sampled analyte level of the user, for transmission to the primary receiver unit 404 via the communication link 403. In certain embodiments, the sensor 401 or the data processing unit 402 or a combined sensor/data processing unit may be wholly implantable under the skin surface of the user.

In some cases, the primary receiver unit 404 may include an analog interface section including an RF receiver and an antenna that is configured to communicate with the data processing unit 402 via the communication link 403, and a data processing section for processing the received data from the data processing unit 402 including data decoding, error detection and correction, data clock generation, data bit recovery, etc., or any combination thereof.

In operation, the primary receiver unit 404, in certain embodiments, is configured to synchronize with the data processing unit 402 to uniquely identify the data processing unit 402 based on, for example, an identification information of the data processing unit 402, and thereafter, to periodically receive signals transmitted from the data processing unit 402 associated with the monitored analyte levels detected by the sensor 401.

Referring again to FIG. 4, the data processing terminal 405 may include a personal computer, a portable computer including a laptop or a handheld device (e.g., a personal digital assistant (PDA), a telephone including a cellular phone (e.g., a multimedia and Internet-enabled mobile phone including an iPhone™, a Blackberry®, or similar phone), an mp3 player (e.g., an iPOD™, etc.), a pager, and the like), and/or an active agent delivery device as described above, each of which may be configured for data communication with the receiver via a wired or a wireless connection. Additionally, the data processing terminal 405 may further be connected to a data network for storing, retrieving, updating, and/or analyzing data corresponding to the detected analyte level of the user.

The data processing terminal 405 may include an active agent delivery device, such as an insulin infusion pump or the like, which may be configured to administer insulin to the user, and which may be configured to communicate with the primary receiver unit 404 for receiving, among others, the measured analyte level. Alternatively, the primary receiver unit 404 may be configured to integrate an infusion device therein so that the primary receiver unit is configured to administer an appropriate active agent (e.g., insulin) to a user, for example, for administering and modifying basal profiles, as well as for determining appropriate boluses for administration based on, among others, the detected analyte levels received from the data processing unit 402. An active agent delivery device may be an external device or an internal device (e.g., wholly implantable in a user).

In certain embodiments, the data processing terminal 405, which may include an active gent delivery device, e.g., an insulin pump, may be configured to receive the analyte signals from the data processing unit 402, and thus, incorporate the functions of the primary receiver unit 404 including data processing for managing the user's insulin therapy and analyte monitoring. In certain embodiments, the communication link 403, as well as one or more of the other communication interfaces shown in FIG. 4, may use one or more of: an RF communication protocol, an infrared communication protocol, a Bluetooth enabled communication protocol, an 802.11x wireless communication protocol, or an equivalent wireless communication protocol which would allow secure, wireless communication of several units (for example, per Health Insurance Portability and Accountability Act (HIPPA) requirements), while avoiding potential data collision and interference.

User input and/or interface components may be included or a data processing unit may be free of user input and/or interface components. In certain embodiments, one or more application-specific integrated circuits (ASIC) may be used to implement one or more functions or routines associated with the operations of the data processing unit (and/or receiver unit) using for example one or more state machines and buffers.

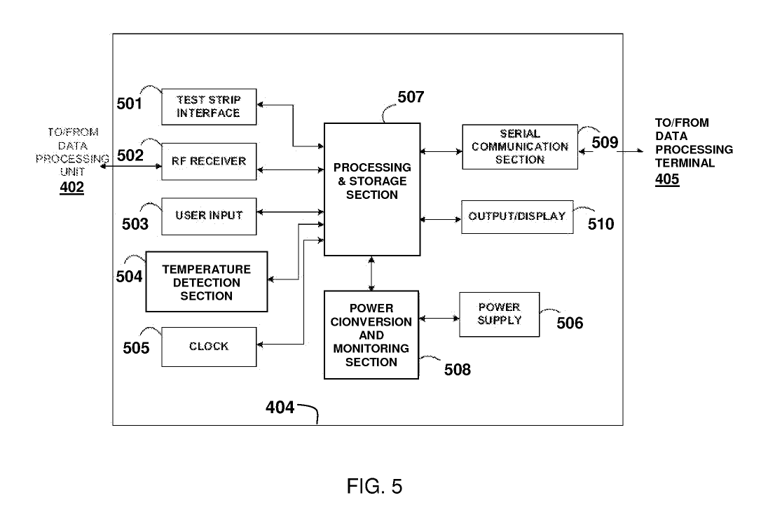

FIG. 5 shows a block diagram of an embodiment of the primary receiver unit 404 of the analyte monitoring device shown in FIG. 4. The primary receiver unit 404 may also include one or more of: a test strip interface 501, an RF receiver 502, a user input 503, a temperature detection section 504, and a clock 505, each of which is operably coupled to a processing and storage section 507. The primary receiver unit 404 also includes a power supply 506 operably coupled to a power conversion and monitoring section 508. Further, the power conversion and monitoring section 508 is also coupled to the processing and storage section 507. Moreover, also shown are a receiver serial communication section 509, and an output 510, each operably coupled to the processing and storage section 507. The primary receiver unit 404 may include user input and/or interface components or may be free of user input and/or interface components.

In further embodiments, the data processing unit 402 and/or the primary receiver unit 404 and/or the secondary receiver unit 406, and/or the data processing terminal/infusion device 405 may be configured to receive the blood glucose value wirelessly over a communication link from, for example, a blood glucose meter. In further embodiments, a user manipulating or using the analyte monitoring device 400 (see FIG. 4) may manually input the blood glucose value using, for example, a user interface (for example, a keyboard, keypad, voice commands, and the like) incorporated in one or more of the data processing unit 402, the primary receiver unit 404, secondary receiver unit 406, or the data processing terminal/infusion device 405.

Additional detailed descriptions are provided in U.S. Pat. Nos. 5,262,035; 5,264,104; 5,262,305; 5,320,715; 5,593,852; 6,175,752; 6,650,471; 6,746,582, and in U.S. Application Publication No. 2004/0186365, the disclosures of each are incorporated herein by reference in their entirety.

### Sensor Control Unit

The sensor control unit can be integrated in the sensor, part or all of which is subcutaneously implanted or it can be configured to be placed on the skin of a user. The sensor control unit is optionally formed in a shape that is comfortable to the user and which may permit concealment, for example, under a user's clothing. The thigh, leg, upper arm, shoulder, or abdomen are convenient parts of the user's body for placement of the sensor control unit to maintain concealment. However, the sensor control unit may be positioned on other portions of the user's body. Embodiments of the sensor control unit may have a thin, oval shape to enhance concealment. Other shapes and sizes may be used.

The particular profile, as well as the height, width, length, weight, and volume of the sensor control unit may vary and depends, at least in part, on the components and associated functions included in the sensor control unit. In general, the sensor control unit includes a housing typically formed as a single integral unit that rests on the skin of the user. The housing typically contains most or all of the electronic components of the sensor control unit.

The housing of the sensor control unit may be formed using a variety of materials, including, for example, plastic and polymeric materials, particularly rigid thermoplastics and engineering thermoplastics. Suitable materials include, for example, polyvinyl chloride, polyethylene, polypropylene, polystyrene, ABS polymers, and copolymers thereof. The housing of the sensor control unit may be formed using a variety of techniques including, for example, injection molding, compression molding, casting, and other molding methods. Hollow or recessed regions may be formed in the housing of the sensor control unit. The electronic components of the sensor control unit and/or other items, including a battery or a speaker for an audible alarm, may be placed in the hollow or recessed areas.

The sensor control unit is typically attached to the skin of the user, for example, by adhering the sensor control unit directly to the skin of the user with an adhesive provided on at least a portion of the housing of the sensor control unit which contacts the skin or by suturing the sensor control unit to the skin through suture openings in the sensor control unit.

When positioned on the skin of a user, the sensor and the electronic components within the sensor control unit are coupled via conductive contacts. The one or more working electrodes, counter electrode (or counter/reference electrode), optional reference electrode, and optional temperature probe are attached to individual conductive contacts. For example, the conductive contacts are provided on the interior of the sensor control unit. Embodiments of the sensor control unit may have the conductive contacts disposed on the exterior of the housing. The placement of the conductive contacts is such that they are in contact with the contact pads on the sensor when the sensor is properly positioned within the sensor control unit.

### Sensor Control Unit Electronics

In certain embodiments, the sensor control unit also includes at least a portion of the electronic components that operate the sensor and the analyte monitoring device system. The electronic components of the sensor control unit may include a power supply for operating the sensor control unit and the sensor, a sensor circuit for obtaining signals from and operating the sensor, a measurement circuit that converts sensor signals to a desired format, and a processing circuit that may be configured to obtain signals from the sensor circuit and/or measurement circuit and provides the signals to an optional transmitter. In some embodiments, the processing circuit partially or completely evaluates the signals from the sensor and conveys the resulting data to the optional transmitter and/or activate an optional alarm system if the analyte level exceeds a threshold or is outside a desired range. The processing circuit may include a digital logic circuitry.

The sensor control unit may optionally contain a transmitter for transmitting the sensor signals or processed data from the processing circuit to a receiver/display unit; a data storage unit for temporarily or permanently storing data from the processing circuit; a temperature probe circuit for receiving signals from and operating a temperature probe; a reference voltage generator for providing a reference voltage for comparison with sensor-generated signals; and/or a watchdog circuit that monitors the operation of the electronic components in the sensor control unit.

In addition to a transmitter, an optional receiver may be included in the sensor control unit. In some cases, the transmitter is a transceiver, operating as both a transmitter and a receiver. The receiver may be used to receive calibration data for the sensor. The calibration data may be used by the processing circuit to correct signals from the sensor. This calibration data may be transmitted by the receiver/display unit or from some other source such as a control unit in a health care provider's office. In addition, the optional receiver may be used to receive a signal from the receiver/display units to direct the transmitter, for example, to change frequencies or frequency bands, to activate or deactivate the optional alarm system and/or to direct the transmitter to transmit at a higher rate.

Moreover, the sensor control unit may also include digital and/or analog components utilizing semiconductor devices, including transistors. To operate these semiconductor devices, the sensor control unit may include other components including, for example, a bias control generator to correctly bias analog and digital semiconductor devices, an oscillator to provide a clock signal, and a digital logic and timing component to provide timing signals and logic operations for the digital components of the circuit.

As an example of the operation of these components, the sensor circuit and the optional temperature probe circuit provide raw signals from the sensor to the measurement circuit. The measurement circuit converts the raw signals to a desired format, using for example, a current-to-voltage converter, current-to-frequency converter, and/or a binary counter or other indicator that produces a signal proportional to the absolute value of the raw signal. This may be used, for example, to convert the raw signal to a format that can be used by digital logic circuits. The processing circuit may then, optionally, evaluate the data and provide commands to operate the electronics.

A threshold value is exceeded if the data point has a value that is beyond the threshold value in a direction indicating a particular condition. For example, a data point which correlates to a glucose level of 200 mg/dL exceeds a threshold value for hyperglycemia of 180 mg/dL, because the data point indicates that the user has entered a hyperglycemic state. As another example, a data point which correlates to a glucose level of 65 mg/dL exceeds a threshold value for hypoglycemia of 70 mg/dL because the data point indicates that the user is hypoglycemic as defined by the threshold value. However, a data point which correlates to a glucose level of 75 mg/dL would not exceed the same threshold value for hypoglycemia because the data point does not indicate that particular condition as defined by the chosen threshold value.

An alarm may also be activated if the sensor readings indicate a value that is beyond a measurement range of the sensor. For glucose, the physiologically relevant measurement range is typically 30-400 mg/dL, including 40-300 mg/dL and 50-250 mg/dL, of glucose in the interstitial fluid.

The alarm system may also, or alternatively, be activated when the rate of change or acceleration of the rate of change in analyte level increase or decrease reaches or exceeds a threshold rate or acceleration. For example, in the case of a subcutaneous glucose monitor, the alarm system might be activated if the rate of change in glucose concentration exceeds a threshold value which might indicate that a hyperglycemic or hypoglycemic condition is likely to occur.

A system may also include system alarms that notify a user of system information such as battery condition, calibration, sensor dislodgment, sensor malfunction, etc. Alarms may be, for example, auditory and/or visual. Other sensory-stimulating alarm systems may be used including alarm systems which heat, cool, vibrate, or produce a mild electrical shock when activated.

### Methods

As disclosed herein, the methods of the present disclosure are useful in connection with a device that is used to determine the amount of an active agent to be delivered to a subject, such as any such device described herein. These methods may also be used in connection with a device that is used to measure or monitor an analyte, including glucose, lactate, oxygen, carbon dioxide, proteins, drugs, or another moiety of interest, or any combination thereof, found in bodily fluid (e.g., blood), including subcutaneous fluid, dermal fluid (e.g., sweat, tears, and the like), interstitial fluid, or other bodily fluid of interest, for example, or any combination thereof.

According to embodiments of the present disclosure, a method for delivering a predetermined amount of an active agent to a subject is provided. In certain instances, the method includes detecting the number of insoluble active agent particles in a fluid flowing through a detector. As described above, the detecting may include receiving signals from a detector configured to detect the number of insoluble active agent particles in a fluid flowing by the detector. In some cases, the detecting includes detecting the size of the insoluble active agent particles in the fluid flowing by the detector. The detecting may also include detecting the number and size of the insoluble active agent particles in the fluid flowing by the detector.

As the insoluble active agent particles flow by the detector, the presence of the insoluble active agent particles in the sensing area of the detector may cause a transient change in an electrical characteristic or an optical characteristic of the fluid flowing by the detector. In certain embodiments, the detecting includes measuring a transient change in an electrical characteristic of the fluid flowing through the detector. As described above, the electrical characteristic may include, but is not limited to, the capacitance, resistance, conductance, impedance, and the like, of the fluid flowing by the detector. In certain instances, the detecting includes measuring a transient change in an optical characteristic of the fluid flowing by the detector. As described above, the optical characteristic may include, but is not limited to, the fluorescence of the insoluble active agent particles, the scattering or blocking of electromagnetic radiation by the insoluble active agent particles in the fluid flowing by the detector, and the like.

In certain embodiments, the method also includes determining the amount of the active agent to be delivered to the subject based on the number of insoluble active agent particles. The determining may include calculating the amount of the active agent based on, for example, the number of insoluble active agent particles, the amount of active agent contained in each particle, and the like. Aspects of the subject method may also include determining the amount of active agent to be delivered to the subject based on the number and size of the insoluble active agent particles. For example, the determining may include calculating the amount of the active agent based on the number of insoluble active agent particles, the size in the insoluble active agent particles, the amount of active agent contained in each particle based on the size of the insoluble active agent particles, and the like.

In certain embodiments, the method further includes adjusting the flow rate of the fluid such that a desired amount of the active agent is delivered to the subject. In some instances, the desired amount of the active agent to be delivered to the subject is predetermined, such as to counteract a high or low level of an analyte in response to signals from one or more sensors, as described in detail above. In addition, it may be desirable to deliver the active agent to the subject over a desired period of time. In these cases, the adjusting includes determining the flow rate of the active agent at a first time point. The flow rate may be determined based on the number and/or size of insoluble active agent particles that flow by the detector over a period of time. Based on the flow rate of the active agent detected at the first time point, the flow rate of the active agent may be adjusted, e.g., increased or decreased as necessary, such that the desired amount of the active agent is delivered to the subject in the desired period of time.

An assessment of the flow rate of the active agent that is obtained via any of the devices and methods of the present disclosure may be used as a basis for adjusting the flow of the fluid carrying the active agent, or adjusting a device, such as a pump, that generates the flow of the fluid carrying the active agent in the flow path, by any suitable means or methods. Such an adjustment may include any of those described in U.S. Pat. No. 6,582,393 and U.S. Application Publication No. 2004/0019321, the disclosures of which are incorporated herein by reference in their entirety. For example, when the fluid is delivered in a pulsed manner, such as via discrete periodic pulses of nominally identical volumes of fluid, and an assessment of the flow rate shows the number of insoluble active agent particles delivered to the subject to be 5% greater than the nominal volume, the pulse period may be decreased by 5% so as to reestablish the intended overall flow rate, or delivery rate, or some other fluid delivery or pulse parameter may be controlled or adjusted to obtain desirable results. For example, when the fluid in the flow path is delivered in a continuous manner, such as in a continuous flow via gravity or via pressure from a source of fluid, such as a reservoir or a bladder, that is controlled, such as via opening or closing or adjusting of a control device, such as a valve, and an assessment of the flow rate shows the number of insoluble active agent particles delivered to the subject to be 5% less than the nominal volume, the control device may be adjusted so as to increase the flow rate to obtain desirable results.

In certain embodiments, the adjusting includes the adjustment of the fluid delivery device, or the fluid flow there from, based on the flow rate of the insoluble active agent particles, or no adjustment of the fluid delivery device where such is undesirable or unnecessary. The adjusting may include providing the flow rate of the active agent to a user, who could then adjust the pump, if and as may be appropriate, such as via a user interface, in any appropriate manner, or automatically adjusting the pump, such as via an automated feedback loop, in any appropriate manner. The adjusting of the fluid delivery device may take place at any suitable time, such as before or at the time fluid delivery from the device commences, or at any time during fluid delivery from the device. Further information concerning control of a fluid delivery device may be found in U.S. Application Publication No. 2005/0235732, the disclosure of which is incorporated herein by reference in its entirety.

### Kits

Kits that include the subject devices useful in practicing the subject methods are also provided. In certain embodiments, a kit for delivering a predetermined amount of an active agent to a subject is provided. The kit may include a device for delivering an amount of an active agent to a subject, as described above, e.g., a device that includes a detector configured to detect the number and/or size of insoluble active agent particles in a fluid flowing by the detector, and a controller operably connected to the detector and configured to determine the amount of the active agent delivered to the subject based on the number and/or size of insoluble active agent particles flowing by the detector.

In addition, the kit may include a fluid that includes insoluble active agent particles. The subject kits may include an active agent delivery device that includes the insoluble active agent particles. For example, the kit may include one or more active agent delivery devices as described herein, and/or other structure that includes the insoluble active agent particles to be used with the active agent delivery device. The insoluble active agent particles may be included in a fluid, which may be included in a reservoir as described in detail above. In some instances, the fluid that includes the insoluble active agent particles is included in a separate container. The separate container may be configured to fill or refill the reservoir as desired. For example, the separate container may include a syringe.

In certain embodiments, the kit may include an active agent delivery device, an infusion set and a housing for containing an active agent and controlling the delivery of the active agent to a subject. For example, an insulin pump may be included in the kit, the insulin pump including an infusion set and in fluid communication with a reservoir having the active agent. Embodiments of the kit may also include an analyte sensor (e.g., glucose sensor) such as a continuous analyte sensor and/or a sensor insertion device and/or a transmitter and/or a receiver.

Embodiments of the subject kits may also include written instructions for using the subject devices. The instructions may be printed on a substrate, such as paper or plastic, etc. As such, the instructions may be present in the kits as a package insert, in the labeling of the container of the kit or components thereof (i.e., associated with the packaging or sub-packaging) etc. In some embodiments, the instructions are present as an electronic storage data file present on a suitable non-transient computer readable storage medium, e.g., CD-ROM, DVD-ROM, BD-ROM, flash memory, diskette, etc. In certain embodiments, the actual instructions are not present in the kit, but means for obtaining the instructions from a remote source, e.g. via the Internet, are provided. An example is a kit that includes a website address where the instructions can be viewed and/or from which the instructions can be downloaded. As with the instructions, this means for obtaining the instructions is recorded on a suitable substrate.

In some embodiments of the subject kits, the components of the kit are packaged in a kit containment element to make a single, easily handled unit, where the kit containment element, e.g., box or analogous structure, may or may not be an airtight container, e.g., to further preserve the contents of the kit until use.

### Utility

Any of the devices and methods described herein may be used to assess, measure, and/or monitor the flow of any fluid that includes, or has added to it, insoluble active agent particles, such as a fluid suspension of insoluble active agent particles. Any such devices and methods may be used to assess, measure, and/or monitor such flow of any magnitude and of any flow characteristic, such as a type of flow, such as continuous or non-continuous flow, an example of the latter being pulsed flow, a direction of flow, a current or force associated with or influencing flow, and the like, and any combination thereof. For example, any such devices and methods may be used to assess, measure, and/or monitor pulsed flow to ascertain whether or not each pulse of fluid has a suitable number of insoluble active agent particles, such as whether or not each pulse of fluid includes substantially the same number of insoluble active agent particles.

Any such devices and methods may be used to assess, measure, and/or monitor an impediment to a desirable or an intended delivery of a fluid that includes insoluble active agent particles. For example, an impediment to the delivery of a fluid that includes insoluble active agent particles may include a gaseous bubble, such as an air bubble, that interferes with such delivery. The bubble may displace the fluid so that the delivery of the fluid that includes insoluble active agent particles is less than what would otherwise be delivered. The presence of such a bubble may significantly or detectable affect an electrical characteristic of the fluid flowing by the detector, such as the capacitance, resistance, conductance, impedance, and the like. The presence of such a bubble may also significantly or detectably affect an optical characteristic of the fluid flowing by the detector, such as the fluorescence, scattering of electromagnetic radiation, blocking of electromagnetic radiation, and the like. Changes in an electrical or an optical characteristic of the fluid may be useful in assessing whether such a bubble is present in the flow path of, or from, a fluid delivery device.

According to a certain embodiments, the subject device and method may have useful application in the delivery of a drug or medicament to a subject, such as the automated delivery of such a drug or medicament to a subject. For example, the subject device and method may find use in the precise automated delivery of such a drug or medicament to a subject, with optional feedback control, such as automated feedback control. The subject device and method may be used in connection with an analyte-detecting device, such as an implantable analyte-detecting device, and an associated drug delivery device that is provided in a patch that may be worn by a subject, such as an adhesive patch that may be affixed to the skin of a subject. For instance, the subject device and method may be used in connection with any of the devices and methods disclosed in U.S. Application Publication No. 2006/0224141, the disclosure of which is incorporated herein by reference in its entirety. For example, the analyte-detecting device may include a glucose-detecting device, the drug delivery device may include an insulin delivery device, the subject may be afflicted with diabetes, and the device and method may be associated with a flow channel of the active agent delivery device, a natural flow channel of the subject, or any suitable flow path, as described herein. In addition, the subject device and method may be used in connection with any of the devices and methods associated with an in vivo FreeStyle™ Navigator™ glucose monitoring device (Abbott Diabetes Care, Alameda, Calif.), based on or related to several U.S. patents and patent applications, such as, U.S. Pat. Nos. 6,175,752, 6,329,161, 6,560,471, 6,579,690, 6,654,625, 6,514,718, 6,605,200, 6,605,201, and 6,932,894, and U.S. Application Publication Nos. 2005/0173245 and 2005/0215871, the disclosures of which are incorporated herein by reference in their entirety.

Each of the various references, presentations, publications, provisional and/or non-provisional U.S. patent applications, U.S. patents, non-U.S. patent applications, and/or non-U.S. patents that have been identified herein, is incorporated herein by reference in its entirety.

Other embodiments and modifications within the scope of the present disclosure will be apparent to those skilled in the relevant art. Various modifications, processes, as well as numerous structures to which the embodiments of the invention may be applicable will be readily apparent to those of skill in the art to which the invention is directed upon review of the specification. Various aspects and features of the invention may have been explained or described in relation to understandings, beliefs, theories, underlying assumptions, and/or working or prophetic examples, although it will be understood that the invention is not bound to any particular understanding, belief, theory, underlying assumption, and/or working or prophetic example. Although various aspects and features of the invention may have been described largely with respect to applications, or more specifically, medical applications, involving diabetic humans, it will be understood that such aspects and features also relate to any of a variety of applications involving non-diabetic humans and any and all other animals. Further, although various aspects and features of the invention may have been described largely with respect to applications involving partially implanted sensors, such as transcutaneous or subcutaneous sensors, it will be understood that such aspects and features also relate to any of a variety of sensors that are suitable for use in connection with the body of an animal or a human, such as those suitable for use as fully implanted in the body of an animal or a human. Finally, although the various aspects and features of the invention have been described with respect to various embodiments and specific examples herein, all of which may be made or carried out conventionally, it will be understood that the invention is entitled to protection within the full scope of the appended claims.

That which is claimed is:

1. A device for delivering a predetermined amount of an active agent to a subject, the device comprising:
a detector configured to detect the number of insoluble active agent particles in a fluid flowing by the detector; and
a controller operably connected to the detector and configured to determine the amount of the active agent to be delivered to the subject based on the number of insoluble active agent particles flowing by the detector.

2. The device of claim 1, wherein the detector is configured to detect the size of the insoluble active agent particles, and wherein the controller is configured to determine the amount of the active agent to be delivered to the subject based on the number and size of insoluble active agent particles flowing by the detector.

3. The device of claim 1, wherein the active agent comprises insulin.

4. The device of claim 3, wherein the insulin comprises acylated insulin.

5. The device of claim 3, wherein the insulin comprises microcrystalline insulin.

6. The device of claim 3, wherein the insulin comprises microspherical insulin.

7. The device of claim 1, wherein the detector is configured to detect an electrical characteristic of the fluid flowing by the detector.

8. The device of claim 7, wherein the electrical characteristic comprises the capacitance of the fluid flowing by the detector.

9. The device of claim 7, wherein the electrical characteristic comprises the resistance of the fluid flowing by the detector.

10. The device of claim 7, wherein the electrical characteristic comprises the impedance of the fluid flowing by the detector.

11. The device of claim 1, wherein the detector is configured to detect an optical characteristic of the fluid flowing by the detector.

12. The device of claim 11, wherein the optical characteristic comprises the fluorescence of the insoluble active agent particles in the fluid flowing by the detector.

13. The device of claim 11, wherein the optical characteristic comprises the scattering of electromagnetic radiation by the insoluble active agent particles in the fluid flowing by the detector.

14. The device of claim 11, wherein the optical characteristic comprises the blocking of electromagnetic radiation by the insoluble active agent particles in the fluid flowing by the detector.

15. The device of claim 1, further comprising:
a reservoir configured to contain a fluid comprising the insoluble active agent particles; and
an infusion set in fluid communication with the reservoir, wherein the infusion set comprises an upstream end and a downstream end.

16. The device of claim 15, wherein the detector is positioned proximal to the upstream end of the infusion set.

17. The device of claim 15, wherein the detector is positioned proximal to the downstream end of the infusion set.

18. The device of claim 15, further comprising a pump in fluid communication with the reservoir and the infusion set.

19. A method for delivering a predetermined amount of an active agent to a subject, the method comprising:
detecting the number of insoluble active agent particles in a fluid flowing by a detector; and
determining the amount of the active agent to be delivered to the subject based on the number of insoluble active agent particles.

20. The method of claim 19, further comprising:
detecting the size of the insoluble active agent particles; and
determining the amount of the active agent to be delivered to the subject based on the number and size of the insoluble active agent particles.

21. The method of claim 19, wherein the active agent comprises insulin.

22. The method of claim 21, wherein the insulin comprises acylated insulin.

23. The method of claim 21, wherein the insulin comprises microcrystalline insulin.

24. The method of claim 21, wherein the insulin comprises microspherical insulin.

25. The method of claim 19, wherein the detecting comprises measuring a transient change in an electrical characteristic of the fluid flowing by the detector.

26. The method of claim 25, wherein the electrical characteristic comprises the capacitance of the fluid flowing by the detector.

27. The method of claim 25, wherein the electrical characteristic comprises the resistance of the fluid flowing by the detector.

28. The method of claim 25, wherein the electrical characteristic comprises the impedance of the fluid flowing by the detector.

29. The method of claim 19, wherein the detecting comprises measuring a transient change in an optical characteristic of the fluid flowing by the detector.

30. The method of claim 29, wherein the optical characteristic comprises the fluorescence of the insoluble active agent particles in the fluid flowing by the detector.

31. The method of claim 29, wherein the optical characteristic comprises the scattering of electromagnetic radiation by the insoluble active agent particles in the fluid flowing by the detector.

32. The method of claim 29, wherein the optical characteristic comprises the blocking of electromagnetic radiation by the insoluble active agent particles in the fluid flowing by the detector.

33. The method of claim 19, further comprising adjusting the flow rate of the fluid such that a desired amount of the active agent is delivered to the subject.

34. A kit for delivering a predetermined amount of an active agent to a subject, the kit comprising:
(a) a device for delivering an amount of an active agent to a subject, the device comprising:
(i) a detector configured to detect the number of insoluble active agent particles in a fluid flowing by the detector; and
(ii) a controller operably connected to the detector and configured to determine the amount of the active agent delivered to the subject based on the number of insoluble active agent particles flowing by the detector; and
(b) a fluid comprising the insoluble active agent particles.
